(19) United States
(12) Patent Application Publication
(10) Pub. No.: US 2005/0123916 A1
(43) Pub. Date: Jun. 9, 2005

(54) Process for the detection of chromosomal aberrations in interphase nuclei

(76) Inventor: Uwe Claussen, Jena (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/492,600

(22) PCT Filed: Oct. 15, 2002
(86) PCT No.: PCT/EP02/11532

(30) Foreign Application Priority Data
Oct. 15, 2001 (EP) 011246006

(51) Int. Cl. C12Q 1/68; C07H 21/04

(52) U.S. Cl. 435/6; 536/25.32

(57) ABSTRACT

The present invention relates to a process for the detection of chromosomal aberrations using e.g. a high resolution multicolor-banding (MCB) technology.

The present invention relates to a process for the detection of chromosomal aberrations in interphase nuclei using a high resolution multicolor-banding (MCB) technology or other techniques which can be used for the visualization of chromosomes in total or in part.

Interphase chromosomes analysed with cytogenetic techniques available at present do not present any recognizable structures such as bands, centromeres, telomeres, or specific shapes. For this reason, it has long been assumed that chromosomes in interphase are relatively decondensed¹. Microirradiation experiments^{2,3} and molecular cytogenetic investigations with whole chromosome paints^{4,5,6} and region specific microdissection probes, however, have been used successfully to improve our understanding of chromosomes in interphase. The concept of the territorial organization of chromosomes in interphase nuclei, originally proposed by Rabl⁷, has been elegantly confirmed^{8,9}.

Until now, however, the structure of chromosomes in interphase nuclei has not been well understood, due mainly to technical problems in the visualization of whole chromosomes in interphase nuclei. The only fluorescence in situ hybridization (FISH) technique available, so far, to characterize the fine structure of human chromosomes at high resolution is the multicolour banding (MCB) technique first described by Chudoba et al.¹⁰, which allows the analysis of DNA-specific multicoloured chromosome bands at high resolution. This technique as also other techniques used so far for chromose investigations was applied to metaphase chromosomes. Preparation of metaphase nuclei and metaphase chromosomes involves cultivation of cells and therefore is time consuming and cumbersome.

It was an object of the present invention to provide a possibility to study chromosomal structure and possible aberrations in a fast and reliable way.

This object was solved according to the present invention by a process for the detection of chromosomal aberrations using for example a high resolution multicolor-banding technique wherein the banding pattern and shape of chromosomes in interphase nuclei is determined by hybridizing interphase cells with a multicolor banding probe mixture for the respective chromosome and comparing the banding pattern to a standard pattern of the respective chromosome.

The multicolor banding technique used in accordance with the present invention is as described by Chudoba et al.¹⁰. In the context of the present invention it has been detected that surprisingly interphase cells can also be used for chromosome diagnosis. Therefore, according to the present invention, interphase nuclei can be used directly for cytogenetic analyses. Such analyses can be performed on nuclei that have been prepared as for cytogenetic analysis, i.e. plated on slides, as well as on three dimensionally intact nuclei, e.g. using confocal laser scanning microscopy.

However, instead of using the multicolor banding technique, also other systems that allow visualization of chromosomes can be used. For example labels like gold particles of different sizes can be used. For such particles computer analysis allows a differentiation, such systems are already known in the art.

The concept of the present invention allows for the first time a rapid and easy diagnosis of chromosomal changes that can be connected with disease or other pathological condititions. Using this concept according to the present invention, cytogenetic diagnosis on chromoses in interphase nuclei becomes possible.

In a preferred embodiment of the present invention, the cells may be synchronized before preparation of interphase nuclei.

In accordance with preferred embodiments of the present invention for multicolor banding region specific partial chromosome paints or other labels are used. Such region specific partial chromosome paints are generated e.g. by isolating single chromosomal regions by for example microdissection or by the use of DNA probes or groups of DNA probes like BAC-, YAC-, or PAC-clones and corresponding labelling.

The labels can be coupled directily to the library, for example by using labelled nucleotides for the amplification, or indirectly. For further details on chromosome paints and labels it is again referred to Chudoba et al.¹⁰ as a general description of the method.

In a preferred embodiment of the present invention, five or more different fluorochromes are used as labels.

Especially preferred examples of labels are DEAC, Spectrum Green, Spectrum Orange and Texas Red, preferably directly coupled to a nucleotide, especially dUTP, and Cy5, which is preferably indirectly visualised via biotin-dUTP and avidin-Cy5.

Using the process of the present invention it is possible to determine chromosomal structure and banding for all chromosomes and all cells containing these chromosomes. Preparation of region specific partial chromosome paint for all chromosomes can be performed in the described manner and used on interphase nuclei of cells like for example bone marrow cells or lymphocytes.

The concept and the process of the present invention are further described in the following relating to specific examples and Figures:

Using the high-resolution DNA-based multicolour banding technique (MCB), the banding pattern and shape of human Chromosom 5 in lymphocyte interphase nuclei, and in nuclei of HeLa cells arrested at different phases of the cell cycle have been investigated. Chromosome 5 in interphase nuclei routinely harvested for chromosome preparation is bent and folded, and shows an MCB pattern similar to that of metaphase chromosome 5. This holds true for all stages of the cell cycle. The length of the chromosome axis is comparable to that of a metaphase chromosome at a 600-band resolution. Therefore, the concept of chromosome condensation and decondensation during mitosis must be reassessed. The MCB pattern was used successfully to identify an interstitial deletion on chromosome 5 in bone marrow interphase nuclei, and to detect an interstitial insertion in lymphocyte interphase nuclei. The identification of chromosome aberrations in interphase nuclei may be of fundamental interest in tumour cytogenetics, and in all chromosome analyses in which a rapid diagnosis is essential.

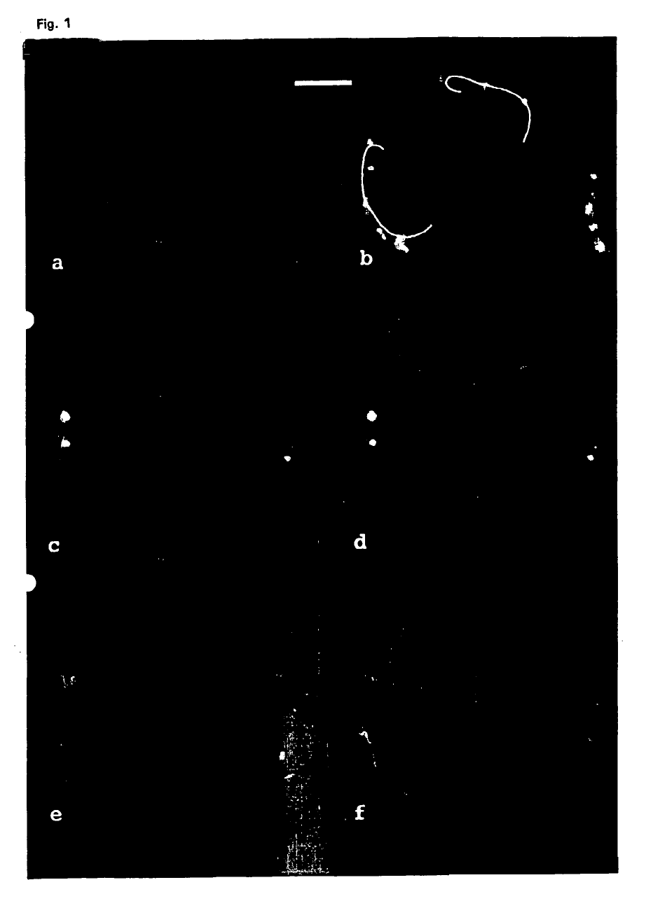

MCB experiments with human chromosome 5-specific hybridization mixture ¹⁰were performed on a total of 106 PHA-stimulated lymphocyte interphase nuclei from a male with a normal karyotype. Nearly all interphase nuclei showed MCB patterns on chromosome 5 very similar to those of corresponding metaphase chromosomes at different coloured band levels (1, 6, 11, 16 and 21; FIG. 1) chosen with the help of the Isis software (MetaSystems, Altlussheim, Germany). Usually, both telomeres were visible and the centromeric region does not show the characteristic constriction.

In 36 of the 106 nuclei (34.0%), the complete MCB patterns (at least 10 coloured bands are present at the 11-coloured-band level, using the Isis software (MetaSystems, Altlussheim, Germany)) of both chromosomes 5 were visible, and in 15 nuclei (14.2%) one completely banded chromosome 5 was visible. One nucleus (0.9%) showed no MCB signal at all. In the remaining 54 nuclei (50.9%), the chromosome 5 MCB patterns were either incomplete, overlapping, or both. The partial loss of chromosome 5-specific MCB signals can be interpreted as mainly due to technical problems.

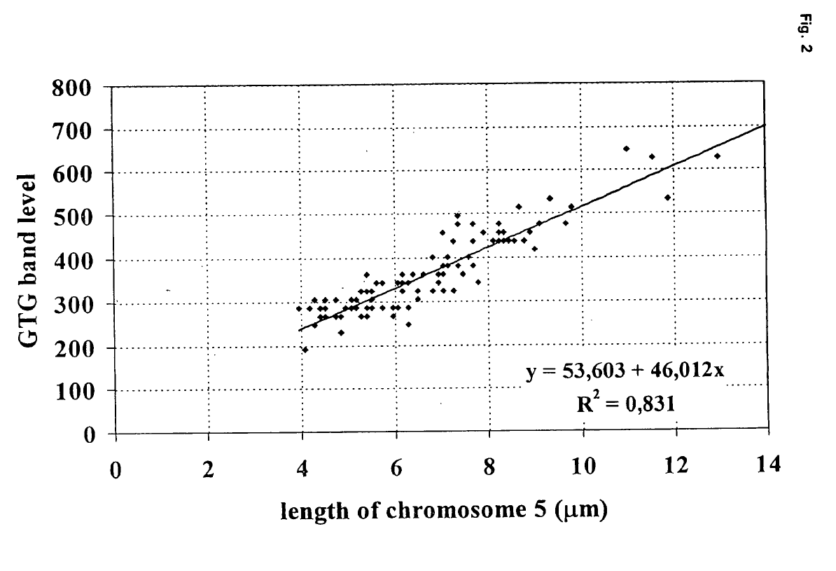

The chromosome axis is defined as the length of the centre line between both telomeres and has been measured on interphase chromosomes painted in one single colour, using the Isis software (FIG. 1). In 67 out of the 87 completely banded interphase chromosomes the axes could precisely be positioned whereas in the remaining 20 chromosomes 0.5 the delineation of the axes was hampered due mainly to strong folding and loop configuration. The length of the interphase chromosomes 5 was found to be 12.0 μm, on average. A standard deviation of 2.3 μm indicates an unexpectedly stable length for chromosome 5 in the interphase nuclei of lymphocytes. For comparison to metaphase chromosomes, 100 metaphase chromosomes 5 of lymphocytes were examined on whether a linear relationships between the length of chromosome 5 and its number of GTG bands exists performing linear regression analyses. The relationship between the variables (p<0.001) was highly significant (FIG. 2) and used to determine the length of interphase chromosomes which were found to be as long as metaphase chromosomes at the 600 band resolution. Interphase chromosomes which were comparable in length to prophase chromosomes as described and proposed by Yunis^{11,12} were not observed.

The reason for this discrepancy may be related to the fact that the process of chromosome preparation leads to a dramatic artificial elongation of chromosomes¹³.

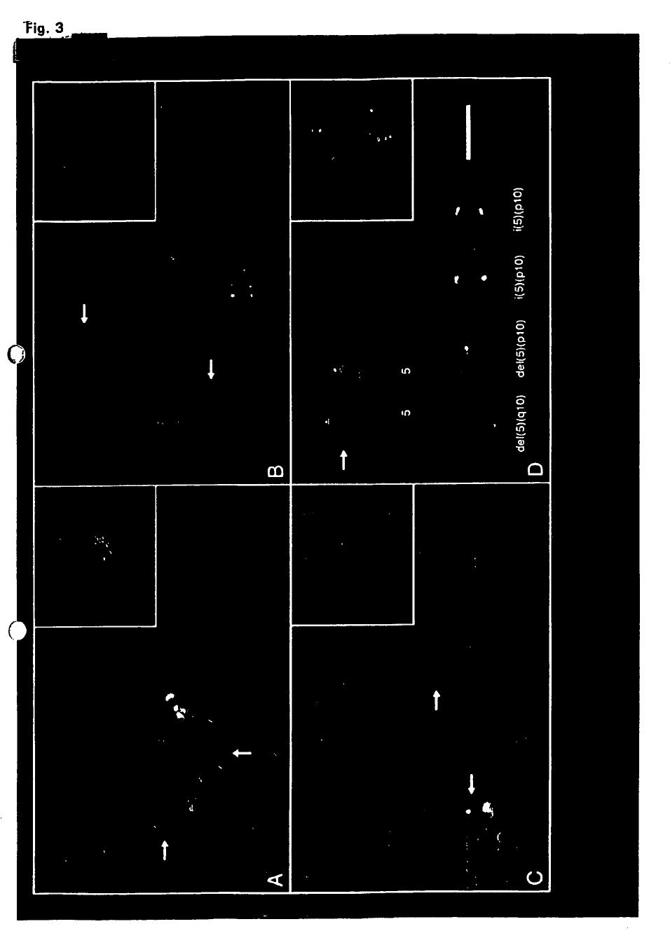

To investigate cell cycle-specific aspects of chromosomal shapes and banding patterns, synchronized HeLa cells were used for hybridization experiments with the MCB probe mixture for chromosome 5. The cells were harvested every two hours throughout the cell cycle, and 50 cells each were analysed during the early, middle, and late G1-phase, the early, middle, and late S-phase, and the early, middle, and late G2-phase. In FIG. 3, representative results are shown which indicate that the MCB pattern of chromosome 5 is present throughout the cell cycle. In contrast to G1, chromosomes in S- and G2-phase are wide, which can be explained simply as a replication-induced difference in the DNA content. Furthermore, the boundaries of chromosomes in S-phase seem to be more diffusely marked compared to those in G1 and G2. It was chosen not to investigate this observation in more detail because such phenomena have been described at the three-dimensional level in relation to the differences between the active and inactive human X chromosomes¹⁴, which were found to have similar volumes but different shapes. Similarly, this pertains also to the positions of active and inactive genes¹⁶ on human chromosomes^{15,16}.

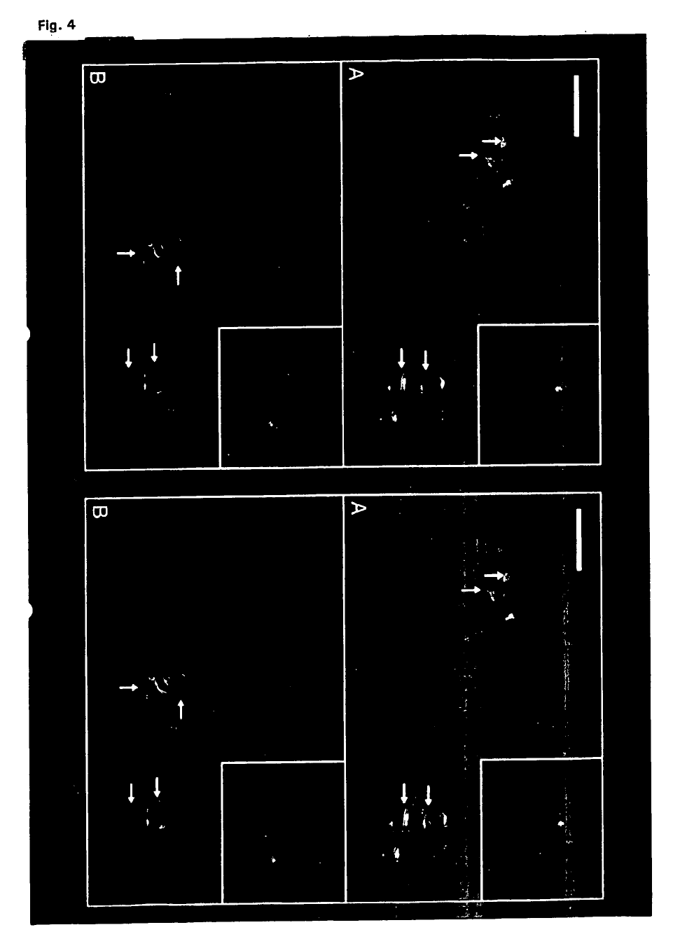

The MCB patterns of interphase chromosomes and their similarity to the DNA-based banded structures of metaphase chromosomes pose the question of whether the MCB pattern of interphase chromosomes can be used for diagnostic purposes. To address this issue, two cases with structural chromosome 5 aberrations, showing a 5q-deletion in cultivated bone marrow cells and a duplicated interstitial insertion in cultivated lymphocytes, respectively, were analysed at interphase. In both cases and in the meantime in some other cases too (data not shown), the aberrations were clearly detectable (FIG. 4) and their breakpoints were confirmed as previously determined on metaphase chromosomes¹⁷. The fact that interphase chromosomes show a banding pattern useful for the detection of chromosome aberrations opens new fields for cytogenetic investigations and routine chromosome analyses.

Chromosomes in interphase are thought to be much longer than in prophase, further condense to metaphase and anaphase chromosomes and become decondensed and longer after telophase. The results in connection with the present invention, however, show that chromosomes in interphase are very simliar in length to metaphase chromosomes.

Therefore, doubts arise about the concept of chromosome condensation in general. Here it is proposed that all the convincing experiments published so far dealing with H3-^{18,19} and SMC-phosphorylation^{20,21} in respect to chromosome condensation may explain phenomena restricted to the formation and/or compaction of chromosome loops, thus influencing the width of chromosomes in two dimensions and probably their volume in three dimensions. The length of the chromosome axis, as the third component of chromosome condensation²², has not been investigated directly by FISH, probably due to technical difficulties. These have been solved here by hybridizing the MCB mixture of chromosome 5 to interphase nuclei.

The following examples and the figures are intended to further illustrate the invention.

### EXAMPLES

### Example 1

Generation of region-specific partial chromosome paints for human chromosome 5.

Region-specific partial chromosome paints for human chromosome 5 were generated by isolating single chromosomal regions by microdissection²³ and subsequent DNA amplification using degenerate oligonucleotide primer (DOP)-PCR^{24,25}. For each chromosomal region-specific library, 8-10 chromosomes were excised. Labelling and signal detection were carried out using five different fluorochromes, of which four were directly coupled to nucleotides (DEAC (NEN-Dupont), Spectrum Green- and Spectrum Orange-dUTP (Vysis), and Texas Red-dUTP (Molecular Probes), and one was used indirectly visualised via biotin-dUTP detected with avidin-Cy5 (Amersham).

### Example 2

Generation of the multicolour banding (MCB) pattern of human chromosome 5.

For generation of the MCB pattern of chromosome 5, the interphase and metaphase cells were hybridized in one step with the MCB probe mixture for chromosome 5, containing two region-specific overlapping chromosomal microdissection libraries for the p-arm and five region-specific overlapping chromosomal microdissection libraries for the q-arm (XCyte 5 mBAND Kit; MetaSystems), for details see Chudoba et al.¹⁰. Hybridization; post-hybridization washes, and signal detection of the region-specific partial chromosome paints, were carried out following standard protocols. Microscopic analysis was performed using an Axioplan II microscope (Carl Zeiss GmbH, Jena) equipped with a CCD camera (Sony), a HBO 100 mercury lamp, and filter sets for DAPI, DEAC, FITC, Cy3, Cy3.5 and Cy5 (Chroma Technologies). Images were captured and analysed using the Isis software package (MetaSystems, Altlussheim, Germany).

### Example 3

The length of chromosome axes and lines, and the determination of the band resolution level.

The lengths of the axes of interphase chromosomes 5 (n=87), the lengths of the lines connecting all midpoints of coloured bands between the telomeres of interphase chromosomes 5 (n=87), and the lengths of GTG-banded metaphase chromosomes 5 (n 100) were measured using the Isis software (MetaSystems, Altlussheim, Germany). The band resolution level was determined on 100 metaphase chromosomes by counting the number of dark and light bands, and comparing the results with the number of bands on the ideograms published in the International System for Cytogenetic Nomenclature (ISCN, 1995)²⁶.

### Example 4

Multicolour banding (MCB) pattern of human Chromosome 5 in HeLa cells at different stages of the cell cycle.

To investigate cell cycle-specific aspects of the chromosomal shapes and banding patterns, synchronized HeLa interphase cells were used for the hybridization experiments with the MCB probe mixture for chromosome 5. Synchronization was achieved after application of a double thymidine block to logarithnücally growing cells and subsequent arrest of the mitotic cells by N₂₀²⁷. In brief, 2 mM thyrnidine was added for 16 h; the cells were then cultured for 11 h in normal medium, and for a further 12 h in medium supplemented with 2 mM thymidine. Eight hours after the removal of the second thymidine block, the cells were treated with N₂O(4.2 kp/cm²) for 4 h. After release of the N₂O block, the mitotic cells were harvested by gentle shaking (mitotic index>90%) and plated into 6-cm petri dishes. After 45 min, the cells had entered into G1-phase. Every 30 min, one petri dish was trypsinized, and the interphase cells were incubated in hypotonic KCl, fixed in three changes of methanol/acetic acid (3:1), dropped onto wet slides, and air dried. In parallel, calyculin A (10 nM) was added to another culture for 60 min, and the degree of synchrony could be roughly estimated in the prematurely condensed chromosomes in the course of the cell cycle.

### Example 5

Detection of structural Chromosome aberrations on human lymphocyte interphase Chromosomes.

Culture of and chromosome preparation from both lymphocytes and bone marrow cells were performed following standard procedures for chromosome analysis. Chromosome structure was analysed in two cases of chromosome 5 aberrations. The first subject, with acute myeloid leukaemia (AML M6), showed an interstitial deletion of 5q in bone marrow interphase nuclei. The second case was a child with congenital malformations, who showed an interstitial duplicated insertion on 5q in lymphocyte interphase nuclei. The MCB analyses were performed with the Isis software (MetaSystems).

List of References:

1. Cornings, D. E. The rationale for an ordered Arrangement of chromatin in the interphase nucleus. Am. J. Hum. Genet. 20, 440-460 (1968).
2. Cremer, T., Cremer, C, Schneider, T., Baumann, H., Hens, L. & Kirsch-Volders, M. Analysis of chromosome position in the interphase nucleus of Chinese hamster cells by laser-UV-microirradiation experiments. Hum. Genet. 61, 201-209 (1982)
3. Cremer, T., Baumann, H., Nakanishi, K. & Cremer, C. Correlation between interphase and metaphase chromosome arrangements as studiert by laser-UV- microbeam experiments. Chrom. Today 8, 201-212 (1984)
4. Cremer, T., Lichter, P., Borden, J., Ward, D. C. & Manuelidis, L. Detection of chromosome aberration in metaphase and interphase tumor cells by in situ hybridization using chromosome-specific library probes. Hum. Genet. 80, 235-246 (1988).
5. Lichter, P., Cremer, T., Borden, J., Manuelidis, L. & Ward, D. C. Delineation of individual human chromosomes in metaphase and interphase cells by in situ suppression hybridization using recombinant DNA libraries. Hum. Genet 80, 224-234 (1988)
6. Pinkel, D., Landegent, J., Collins, C., Fuscoe, J., Segraves, R., Lucas, J. & Gray, J. Fluorescence in situ hybridization with human chromosome-specific libraries detection of trisomy 21 and translocations of chromosome 4. Proc. Natl. Acad. Sci. USA 85,9138-9142 (1988).
7. Rabl, C. Über Zellteilung. Morphologisches Jahrbuch 10, 214-330 (1885)
8. Cremer, T., Kurz, A., Zirbel, R., Dietzel, S., Rinke, B., Schrock, E., Speicher, M. R., Mathieu, U., Jauch, A., Emmerich, B., Schertan, H., Ried, T., Cremer, C. & Lichter, P. Role of chromosome territories in the functional compartmentalization of the cell nucleus. Cold Spring Harb. Symp. 58, 777-792 (1993).
9. Chevret, E., Volpi, E. V. & Sheer, D. Mini review: Form and function in the human interphase chromosome. Cytogenet. Cell Genet. 90, 13-21 (2000)
10. Chudoba, I., Plesch, A., Lörch. T., Lemke, J., Claussen, U. & Senger, G. High resolution multicolor-banding: a new technique for re:fined FISH analysis of human chromosomes. Cytogenet. Cell Genet. 84, 156-160 (1999).
11. Yunis, J. J. High resolution of human chromosomes. Science 191, 1268-1270 (1976)
12. Yunis, J. J. Mid-prophase human chromosomes. The attainment of 2000 bands. Hum Genet. 56, 293-298 (1981),
13. Hliscs, R., Mühlig, P. & Claussen, U. The spreading of metaphase is a slow process which leads to a stretching of chromosomes. Cytogent. Cel Genet. 76, 167-171 (1997).
14. Eils, R., Dietzel, S., Bertin, E., Schrack, F., Speicher, M. R., Ried, T., Robert- Nicoud, M., Cremer, C. & Cremer, T. Three-dimensional reconstruction of painted human interphase chromosomes—active and inactive X-chromosome territories have similar volumes but differ in shape and surface structure. J. Cell Biol. 135, 1427-1440 (1996).
15. Kurz, A., Lampel, S., Nickolenko, J. E., Bradl, J., Brenner, A., Zirbel, R. M., Cremer, T. & Lichter, P. Active and inactive genes localize preferentially in the periphery of chromosome territories. J. Cell Biol. 135 1195-1205 (1996).
16. Dietzel, S., Schiebel, K., Little, G., Edelmann, P., Rappold, G. A., Eils, R., Cremer, C. & Cremer, T. The 3D positioning of ANT2 and ANT3 genes within female X chromosome territories correlates with gene activity. Exp Cell Res. 252, 363-375. 1999)
17. Lemke, J., Chudoba, I., Senger, G., Stumm, M., Loncarevic, Henry; C., Zabel, B., Claussen, U. Improved definition of chromosomal breakpoints using high resolution multicolour banding (MCB). Hum. Genet. (submitted, 2000)
18. Gurley, L. R., D'Anna, J. A., Barhan, S. S., Deaven, L. L. & Tobey, R. A. Histone phosphorylation and chromatin structure during mitosis in Chinese hamster cells. Eur. J. Biochem. 84, 1-15 (1978).
19. Wei, Y., Yu, L., Bowen, J., Gorovsky, M. A. & Allis, C. D. Phosphorylation of histone H3 is required for proper chromosome condensation and segregation. Cell 97, 99-109 (1999).
20. Hirano, T. SMC-mediated chromosome mechanics: a conserved scheme from bacteria to vertebrates? Gen. Develop. 13, 11-19 (1999).
21. Strunnikov, A. V. & Jessberger, R.: Structural maintenance of chromosomes (SM proteins). Eur. J. Biochem. 263, 6-13 (1999)
22. Koshl, D. & Strunnikov, A. Mitotic chromosome condensation. Ann. Rev. Cell Dev. Biol. 12, 305-333 (1996).
23. Senger, G., Ludecke, H.-J., Horsthemke, B. & Claussen, U. Microdissection of banded human chromosomes. Hum. Genet. 84, 507-511 (1990)
24. Senger, G., Friedrich, U., Claussen, U., Tommerup, N. & Broridum-Nielsen, K. Prenatal diagnosis of a half cryptic translocation using chromosome microdissection. Prenat. Diagn. 17, 369-374 (1997).
25. Chudoba, I., Rubtsov; N., Senger, G., Junker, K., Bleck, C. & Claussen, U Improved detection of chromosome 1.6 rearrangements in acute myeloid leukemias using 16p and 16q specific microdissection libraries. Oncot Rep. 3, 829-832 (1996).
26. ISCN (1995): An International System for Human Cytogenetie Nomenclature, Mitelman, F. (ed), S. Karger, Basel, 1995.
27. Schmiady, H., & Sperling, K. Length of human prematurely condensed chromosomes during GO and GI phase. Exp. Cell Res. 134, 461-465 (1986).
   Figure Legends

FIG. 1

Human chromosome 5 in a normal lymphocyte interphase nucleus at different MCB pattern resolution. In a-f, both interphase chromosomes 5 are visible, showing the same coloured patterns as their corresponding metaphase chromosomes 5 on the right. Scale bar, 5 pm. a shows both chromosomes 5 in the interphase nucleus hybridized with the MCB probe mixture. The different colours arise from the fluorescence signals taken with the individual filter combinations. DNA-based pseudocolours are not integrated. b shows both chromosomes 5 in one colour. The MCB probe mixture has been used as a whole chromosome-painting probe. The lines represent the chromosome axes as centrelines between the telomeres which have been used to measure the length of the interphase chromosomes 5. c to f show both chromosomes 5 in 6, 11, 16, and 21 different pseudocolours, respectively, obtained with the help of the Isis software.

FIG. 2

Correlation between the length of metaphase chromosome 5 of lymphocytes (marked as ♦; n=100) and its GTG-band resolution. The regression equation is highly significant (p<0.001) and indicates to a linear relationship between the variables

FIG. 3

Multicoloured human chromosomes 5 in the G1-, S- and G2-phases, and in the metaphase stage of HeLa cells. The light arrows indicate the normal chromosomes 5, the grey arrows indicate the chromosomes 5 with a deletion of the short arm (del(5)(p11)), and the grey triangles indicate the chromosomes 5 with a deletion of the long arm (del(5)(q11)) and the isochromosomes 5p (i(5)(p10)). Scale bar, 5 pm. a shows the MCB pattern of the normal and rearranged chromosomes 5 of a HeLa cell arrested at GI of the cell cycle.

Some chromosomes 5 are close to each other by chance, and the MCB pattern is identical to that of the chromosomes 5 in metaphase (seed). On the right, the same interphase nucleus is shown at a lower magnification. The cell is DAPI-stained (blue background), and the different colours arise from the fluorescence signals taken with the individual filter combinations. In b, an interphase nucleus is shown arrested in S-phase, comparable to a; and in c an interphase nucleus is shown arrested in G2-phase. Chromosome 5 in S-phase is as long as in the G1- and G2-phases, but is wider. In all stages of the cell cycle, the MCB patterns of chromosomes 5 are identical. The forms of the coloured bands, however, differ significantly.

FIG. 4

At the DNA-based MCB level, structural chromosome aberrations are visible in human lymphocyte and bone marrow interphase nuclei. The same cell, which is DAPI-stained, is shown on the top right at lower magnification. The different colours arise from the fluorescence signals taken with the individual filter combinations. The light arrows indicate the breakpoints of the normal chromosomes 5 in the interphase (left) and metaphase (right) stages. The grey arrows indicate the inserted band on the rearranged chromosome 5 in a, and to the deletion breakpoint of the rearranged chromosome 5 (5q) in b Scale bar, 5 pm. a shows lymphocyte chromosomes 5 of a boy with congenital malformations, revealing a duplicated insertion of the green band of 5q31, between the ochre and white bands at 5q13. The green band on the short arm of the chromosome differs from the green colour of band 5q31 and from that of the inserted band, which can be confirmed with the Isis software. b shows a chromosome 5 from the bone marrow of a patient with leukaemia (AML M6) and a 5q-syndrome.

1. Process for the detection of chromosomal aberrations using e.g. a multicolor-banding technique for characterizing chromosomes in such a way that a banding pattern and the shape of at least one chromosome in interphase nuclei are visualized using one or several suitable DNA probes for the respective chromosome via hybridization techniques resulting in a DNA mediated banding pattern.

2. Process according to claim 1, wherein synchronized interphase cells are used.

3. Process according to claim 1, wherein the multicolor banding probe or probe mixture comprises a region-specific partial chromosome paint.

4. Process according to claim 1, wherein the DNA probes are selected from BAC-, PAC- and YAC clones.

5. Process according to claim 3, wherein the partial chromosome part is directly or indirectly labeled.

6. Process according to claim 1, wherein one or more different fluorochromes or other dyes or labels useful for any kind of microscopy examination (e.g. laser scanning microscopy, electron microscopy) are used as labels for the DNA probes.
